(19) United States
(12) Patent Application Publication
(10) Pub. No.: US 2017/0291007 A1
(43) Pub. Date: Oct. 12, 2017

(54) Therapeutic Vibration System

(71) Applicant: SONUSMED, INCORPORATED, Gilbert, AZ (US)

(72) Inventors: Anju Dubey, Gilbert, AZ (US); Shreya Dubey, Gilbert, AZ (US)

(73) Assignee: SONUSMED, INCORPORATED, Gilbert, AZ (US)

(21) Appl. No.: 15/480,179

(22) Filed: Apr. 5, 2017

Related U.S. Application Data
(60) Provisional application No. 62/318,890, filed on Apr. 6, 2016.

(51) Int. Cl. A61M 21/00; G08C 17/02

(52) U.S. Cl. CPC A61M 21/00; G08C 17/02; A61M 2021/0022; A61M 2210/04; A61M 2205/10; A61M 2205/502; A61M 2205/3584; A61M 2205/8206

(57) ABSTRACT

A therapeutic vibrational system for treating such conditions as hypertension may have a plurality of vibrating patches; each including a vibrating motor with an adhesive for application to a person's skin permitting the plurality of vibrating patches to be placed in two or more locations on the person's skin, as well as a controller with a user interface and configured to provide control instructions to the plurality of vibrating patches.

### CROSS REFERENCE TO RELATED APPLICATIONS

This application claims the benefit of priority to U.S. provisional patent application Ser. No. 62/318,890, filed on Apr. 6, 2016; the entirety of which is hereby incorporated by reference herein.

### BACKGROUND

Two of the top killers across the globe, heart disease and stroke have been linked to high blood pressure or hypertension. Although there are various definitions of hypertension, one of the widest accepted is blood pressure above 140/90. It is estimated that globally a billion people have hypertension, which is often called silent killer because most people are not even aware of their high blood pressure. Hypertension is a leading cause of kidney failure, aneurysms and fatal cardiovascular events and contributes to an estimated 1000 deaths per day in United States according to Center for Disease Control (CDC) website. One in three people suffer from hypertension in US, i.e., about 67 million people.

Currently there are many medications available for hypertension, e.g. β-blockers, ACE inhibitors, etc. Physicians start by prescribing “first line” drugs and continue adding prescriptions until the blood pressure reaches a lower level. Despite the current medications, half of the hypertensive people in US (34 million) have not been able to control their hypertension (per the CDC). Because multiple drugs are often required, the chances of drug interaction and side effects increase. Medication regimens, with multiple prescriptions and/or the latest drugs, can also be too expensive for chronic therapy.

### SUMMARY OF THE INVENTION

The present disclosure is directed to a noninvasive device to be used alone or as adjunct therapy with current medications for treatment of hypertension and other stress related conditions such as arrhythmia, PTSD, sleep apnea, depression, and achalasia.

In some embodiments, a therapeutic vibrational system may include a plurality of vibrating patches, each including a vibrating motor and an adhesive for application to a person's skin. The plurality of vibrating patches may be placed in two or more locations on a person's skin. The system may also have a controller with a user interface and configured to provide control instructions to the plurality of vibrating patches. In some embodiments the controller may be a remote controller that produces a wireless signal that may be received by a wireless signal receiver of each of the plurality of vibrating patches. The remote controller can be a mobile device such as a mobile telephone. In some embodiments, the vibrating patches may be connected to a frame, by flexible tethers allowing adjustable placement of the vibrating patches with respect to the frame. The tethers may carry a communication line for transmission of control instructions from said frame controller to the vibrating patches.

In some embodiments, the frame controller includes a user interface and a wireless signal receiver for receiving a wireless signal having control instructions from a remote controller. A remote controller may have a user interface having amplitude control and a frequency control and can be a mobile device or mobile telephone. The frame may have a battery such that the frame can supply electrical power to the plurality of vibrating patches from the battery. The vibrating patches may be connected to the frame by tethers that may be flexible, allowing adjustable placement of the plurality of vibrating patches with respect to the frame. The tethers may have a power line for providing electrical power from frame battery to each of the plurality of vibrating patches. The tethers may provide a communication line for transmission of control instructions from said frame controller to the plurality of vibrating patches. In some embodiments, the frame controller may have a wireless signal receiver for receiving a wireless signal having control instructions from a remote controller that can be a mobile device.

In some embodiments, a cover having an adhesive patch, also may have an opening for receiving one of the vibrating patches. The cover may have a detachably attachable cap having an adhesive patch, wherein the cap includes an opening for detachably attachment to one of said plurality of vibrating patches. In some embodiments, a therapeutic vibrational therapeutic vibrational system includes a frame with a frame controller; a wireless signal receiver for receiving control instructions; and a battery. The system may include a plurality of vibrating patches, each having a vibrating motor and an adhesive for application to a person's skin that permits placement of the vibrating patches in two or more locations on said person's skin. The tethers may be coupled to the vibrating patches and the frame, with each tether having a communication line for transmission of said control instructions from said frame controller to the plurality of vibrating patches and a power line for providing electrical power from the frame battery to each of the plurality of vibrating patches, such that the wireless signal receiver of the frame receives control instructions from a remote controller having a user interface. The remote controller can be a mobile device, particularly a mobile telephone. The remote controller may have a user interface having amplitude control and a frequency control. The therapeutic vibrational system may have at least two and in some embodiments four vibrating patches. The vibrating patches may be operated at frequencies ranging from 20 Hz to 400 Hz with a vibration amplitude of about 0.2 to 10 G (gravitational load or G-force) ranges.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure.

FIG. 1 is a graph showing before and after blood pressure values for volunteers, arranged by their ages.

FIG. 2 shows the first prototype of a non-invasive therapeutic vibrational instrument, with the different tips and a recharging station.

FIG. 3 shows a second prototype that was used for extensive blood pressure testing in large short-term and long-term studies.

FIG. 4 is a graph showing changes in systolic blood pressure with treatment over 10 days, with both hypertensive and hypotensive blood pressures trending toward normal.

FIG. 5 is a bar graph of aggregate blood pressures of hypertensive, pre-hypertensive and normal individuals, in which hypertensive and pre-hypertensive volunteers both experienced significant improvements.

FIG. 6 is a graph showing a correlation between the starting systolic blood pressure and the size of the decrease in blood pressure after treatment.

FIG. 7 shows an exemplary therapeutic vibrational system comprising a frame with a plurality of vibrating patches connected by flexible tethers and a controller.

FIGS. 8A and 8B show an exemplary detachable module detached from the frame (8A) and an exemplary frame being charged by a charger inserted into an electrical outlet (8B).

FIG. 9 shows an exemplary therapeutic vibrational system comprising a frame with a plurality of vibrating patches connected by flexible tethers, as well as a controller.

FIG. 10 shows a back view of the exemplary therapeutic vibrational system shown in FIG. 9, having a frame controller.

FIG. 11 shows an exemplary therapeutic vibrational system comprising a frame with a plurality of vibrating patches connected by flexible tethers.

FIG. 12 shows an exemplary therapeutic vibrational system comprising a frame with a vibrating patch connected by flexible tethers.

FIG. 13 shows an exemplary therapeutic vibrational system comprising a nose frame with a plurality of vibrating patches connected to the nose frame.

FIG. 14 shows a therapeutic vibrational device with a nose frame and only two self-contained patches.

FIG. 15 shows an exemplary patch being inserted into a cover having an opening for receiving the patch and an adhesive patch for attachment to the skin.

FIG. 16 shows an exemplary adhesive patch with a removable adhesive cover.

FIG. 17 shows an exemplary cap being coupled with a vibrating patch and an adhesive patch on the cap for attachment to the skin.

FIG. 18 shows the exemplary cap of FIG. 17 coupled to the vibrating patch with the removable adhesive cover being removed.

FIGS. 19 and 20 show an exemplary vibrating patch having a vibrating motor, a battery, a signal receiver, a signal transmitter and a patch controller.

FIG. 21 shows a plurality of exemplary vibrating patches attached to a person's cheek and behind the ear and a mobile device controller having a control program and user interface.

FIG. 22 shows an exemplary charger for a plurality of vibrating patches.

Corresponding reference characters indicate corresponding parts throughout the several views of the figures. The figures represent an illustration of some of the embodiments of the present disclosure and are not to be construed as limiting the scope of the invention in any manner. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present disclosure.

### DETAILED DESCRIPTION

The inventors know of no previous noninvasive device to treat hypertension. The disclosed devices may be used as an adjunct therapy as well as stand-alone treatment of hypertension as well as other diseases. The noninvasive devices may also represents a novel alternative therapeutic approach, in addition to current medications, life style changes and surgery, to treatment of chronic diseases like hypertension, arrhythmia, sleep apnea, post traumatic stress disorder, depression, achalasia, etc. Such treatment is not just innovative and noninvasive, it may decreased threat of drug interaction, negligible side effects, increased compliance, and may be a safe, economical treatment with enormous humanitarian worldwide value in treating debilitating, even fatal diseases.

The inventors tested various durations, frequencies and amplitudes with volunteers. The first step was a pilot study with a prototype. With that success, they turned to improving on the prototype as discussed below. First, a preliminary test is discussed, then the prototype work and further testing are discussed.

The first step toward the invention was the observation by one of the inventors that humming calmed her before exams. Next the inventors planned and tested humming and OMMM chanting with friends and neighbors volunteering. Encouraged by the findings, they reviewed scientific literature for possible explanations. It was found that the vibrational phonation has several possible routes to affect blood pressure and heart rate.

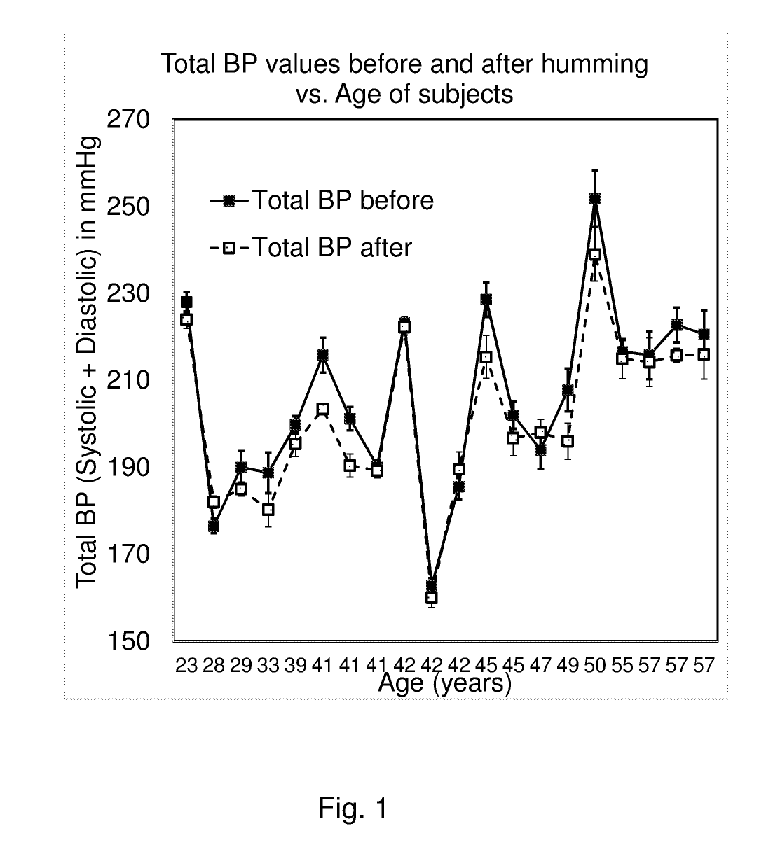

The results are shown in FIG. 1. The graphs with two lines, respectively, which display each volunteer's blood pressure before and after, one over the other. This indicates how variable the results were; often there was a small but positive change; but for many, there was no notable change. Upon further research the inventors found that humming or chanting appeared to cause a vasovagal response in some individuals, with the repeated activity eliciting a coughing reflex. At the least, the induced coughing interrupted the humming or chanting. At the worst, the volunteer ceased humming or chanting: hence, volunteers who coughed also had poor compliance. While these preliminary results were suggestive, the inventors concluded uniform improvements required a more predictable intervention.

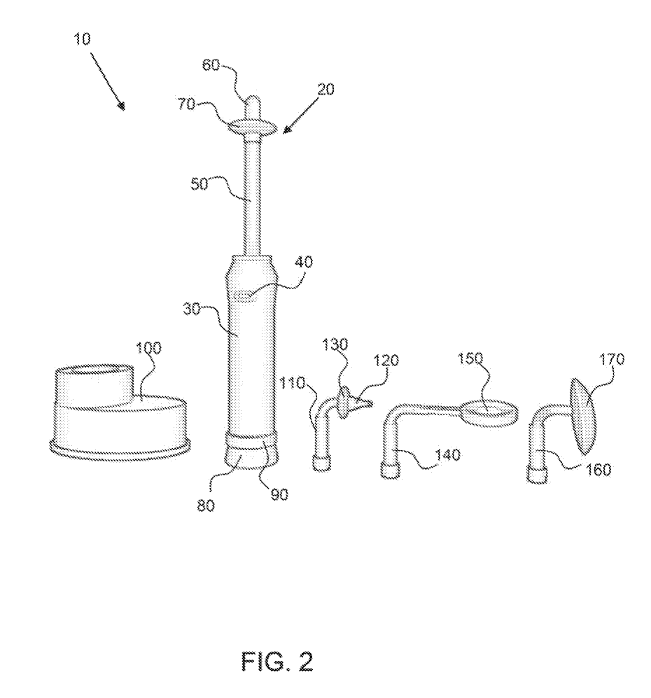

Next the inventors sought the most convenient vibrating object to adapt for furthering investigations—namely, a vibrating toothbrush adapted as shown in FIG. 2 without the brush portion. Because the humming and chanting had an internal effect, the inventors initially tried this device in a body orifice—the nose or ear. There poor volunteer reactions, including a tickle response, were seen.

FIG. 2 is the first attempted therapeutic vibrational device 20. The therapeutic vibrational device 20 may be part of a system 10. The therapeutic vibrational device 20 may have a handle 30, on which there may be a light 40 to indicate the device 20 is on. The device 20 connects to a connector 50 that positions any of a variety of tips for exposure to a controlled vibrational experience. Device 20 is shown with nasal tip 60. Between the nasal tip 60 and the connector 50 a guard 70 may help the operator avoid insertion too deep into the nasal cavity. Device 20 may have a base 80 and ring 90 to enclose the contents of the handle.

Handle 30 that may contain batteries and a microprocessor (not shown) that control operation of the nasal tip 60 with the appropriate power and frequency. The microprocessor is programmed for consistent and controlled durations, frequencies and amplitudes which can be increased or decreased as needed. In some embodiments, the user can adjust these parameters, such as through a rocker on-off switch that also slides to adjust one parameter or another. The microprocessor may control the vibrations within a range of about 20 to 500 Hz and in some embodiments a range of about 100-450 Hz. In some embodiments, there are provided vibrations of about 200 Hz.

Besides the therapeutic vibrational device 20, the system may include a recharging station 100, into which the therapeutic vibrational device with rechargeable batteries is set when not in use. A variety of other tips (besides the nasal tip 60) are shown. Ear tip 120 also may have a guard 130 and a connector post 110 to fit into connector 50. The next tip is a mouth tip 150 that also may have a connector post 140. An additional tip with a concave surface is the tip 170 that is also equipped with connecting post 160; this concave surface is designed to rest comfortably against the side of the throat and/or the cheek overlying the sinus.

Basically all components of FIG. 2 embodiment can have a variety of shapes, as one skilled in the art would refine the designs for esthetics and function. For example, the recharging station 100 is shown to be circular with two levels. Rather than a circle, it can be square, oval, hexagon, etc. In addition, it need not have two levels, but can be designed as one compact unit. The tips may be designed to be compatible for the site occupied (nose, ear or mouth). Not shown is an electrical cord to power the recharging station.

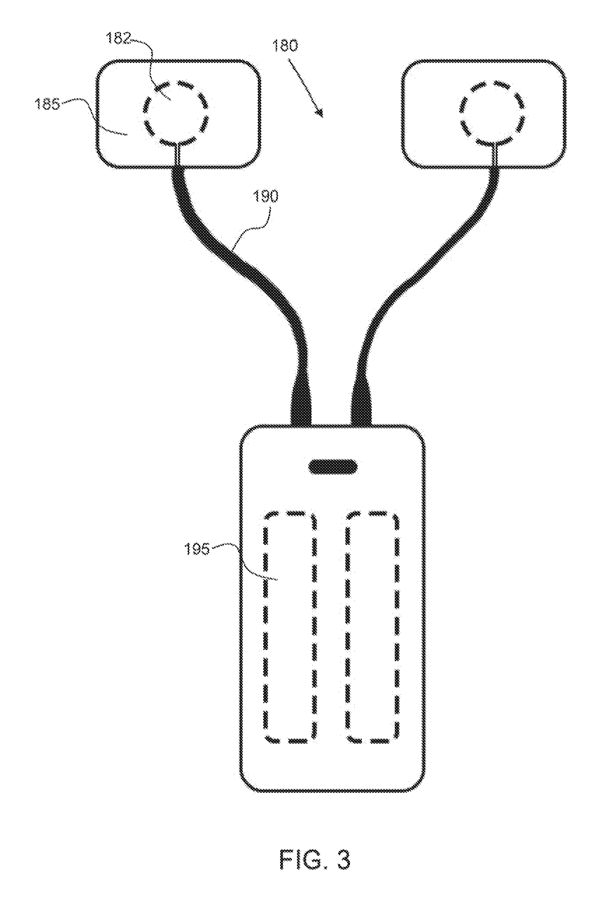

Next a second prototype was designed, as shown in (FIG. 3), and was tested on large numbers of patients. The device 180 had four flat-heads 185 (two are shown) that contain vibrating motors 182 (200 Hz and 2 G vibration amplitude) connected via tethers 190 to batteries 195. Moleskin was used to hold the motors in place. Forms for each patient collected information for the patient's age, gender, family health history, current health issues, medications and approximate weight. The forms also provided space for recording data, including times of treatment, systolic and diastolic blood pressures and heart rate.

Treatments with the prototype included vibrations for various time periods and at different locations on the head and face. Treatment was twice a day, basically morning and afternoon with at least five hours in between. Blood pressure was measured twice before and twice after each treatment. Initial motor placements were on the maxillary area on each side of the nose and around ears. All motors were operated at the same time.

A specific protocol was followed for each treatment, including the patient sitting on a chair for at least 5 min, while the researcher placed the BP monitor at heart level, wrapped it just above the elbow and prepared the device for use. Researcher and patient were advised to not talk during BP readings. Two BP readings were taken initially. Next the vibrating motors were placed on the cheeks and neck, and the researcher operated the motors for selected periods of time (see below) behind the ears. Then the motors were removed and put away, with a delay of at least 2 min until two BP readings were again obtained.

With this embodiment, the inventors initially conducted a short term study to analyze the best placement of the vibrating device on the head and/or face. It was a one-time test on 386 males and females from aged 21-83. Blood pressure and heart rate were taken twice. Then treatment was given for four periods of 30 sec with 5 sec breaks between each. After two min, two additional measurements of blood pressure and heart rate were taken. The vibrations of the device varied from 150-300 Hz and 1.6-7 G amplitude. Even after this short-term study, the average systolic blood pressure decreased from an initial 130 mmHg to about 120 mmHg (P=0.0008 from a two-tailed T-test). After observing and comparing the results, the inventors decided to proceed with vibrations on areas on the cheeks (adjacent the nose) and behind the ears.

A longer-term study of 10 days was performed. This group was treated twice a day with vibration therapy at four locations (cheeks on both sides of the nose and behind/below both ears). Blood pressure was measured twice before and after each treatment. Here, the therapeutic vibrational device was turned on for 30 sec, off for 5 sec and repeated three more times. Some volunteers were hypertensive (above 140 mmHg systolic pressure), and some volunteers were normotensive (about 120 mmHg) and others were hypotensive (less than 110 mmHg). FIG. 4 shows how the systolic pressures converged toward normotensive during the 20 treatments (2 each day).

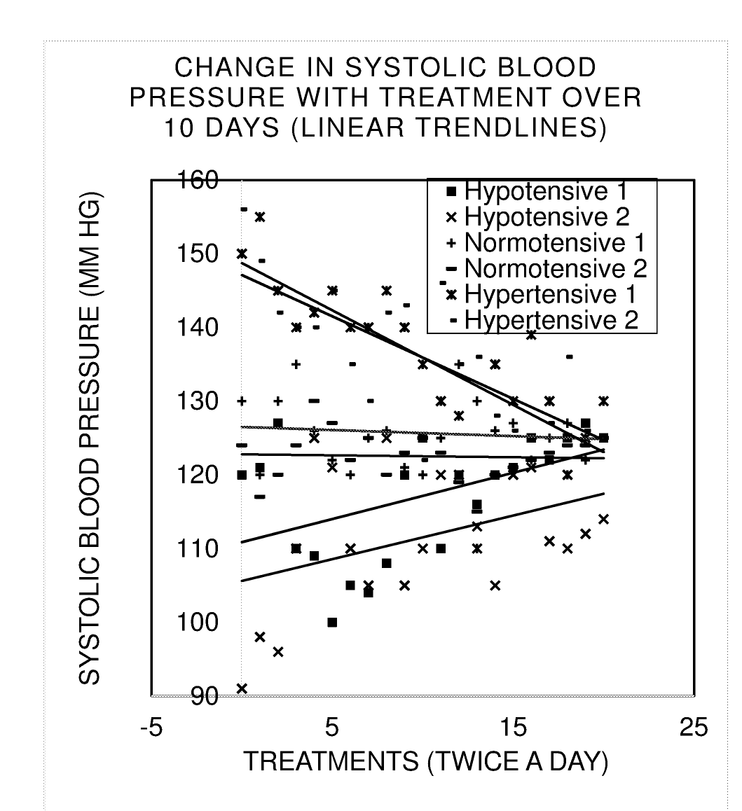

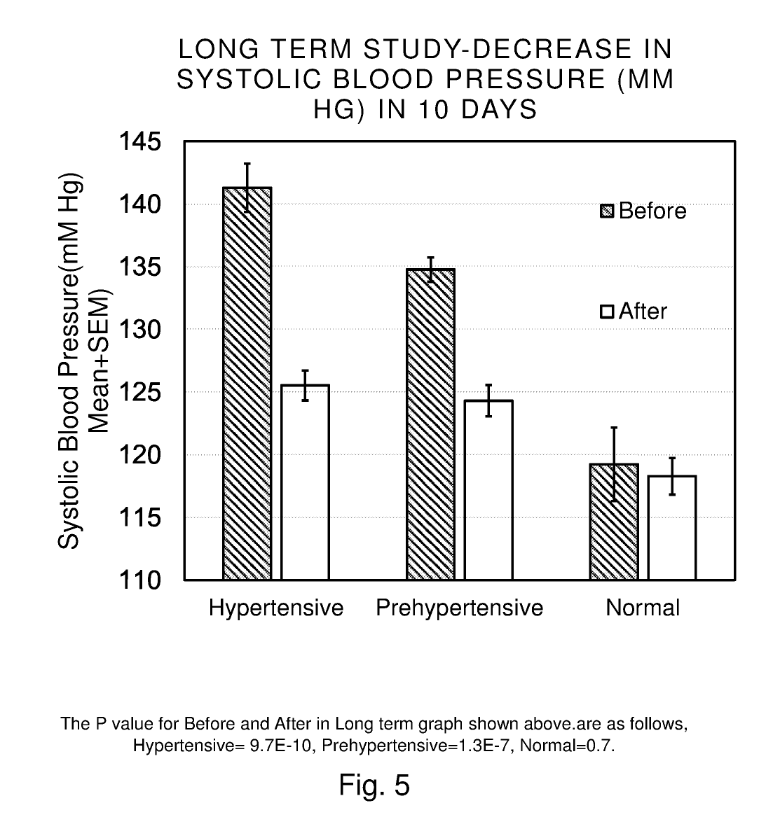

The FIG. 5 bar graph of hypertensive (greater than 140 mmHg systolic), pre-hypertensive (125-140 mmHg) and normal blood pressures (about 120 mmHg) at the start and finish of the 10-day study. These indicate significant improvement for the hypertensive and pre-hypertensive volunteers with minor change for the normal blood pressure individuals. There was a surprising discovery in this study regarding the hypotensive individuals in the study. The three hypotensive volunteers in the long-term study showed a remarkable increase in blood pressure at the end of the 10-day treatment with the second prototype. This observation along with the decrease in blood pressure of hypertensive individuals as well as negligible changes in blood pressure of the normotensive volunteers suggest that this device may stimulate an endogenous resetting mechanism for blood pressure. It also seems to restore normal homeostatic state of the body and only effects changes in blood pressure if needed (hypertensive and hypotensive conditions) and does not affect the normotensive state. This phenomenon provides strong support to the safety of the underlying mechanism of action and underlies the difference of such treatment in contrast with current hypertension medication which cause hypotension as a frequent side effect.

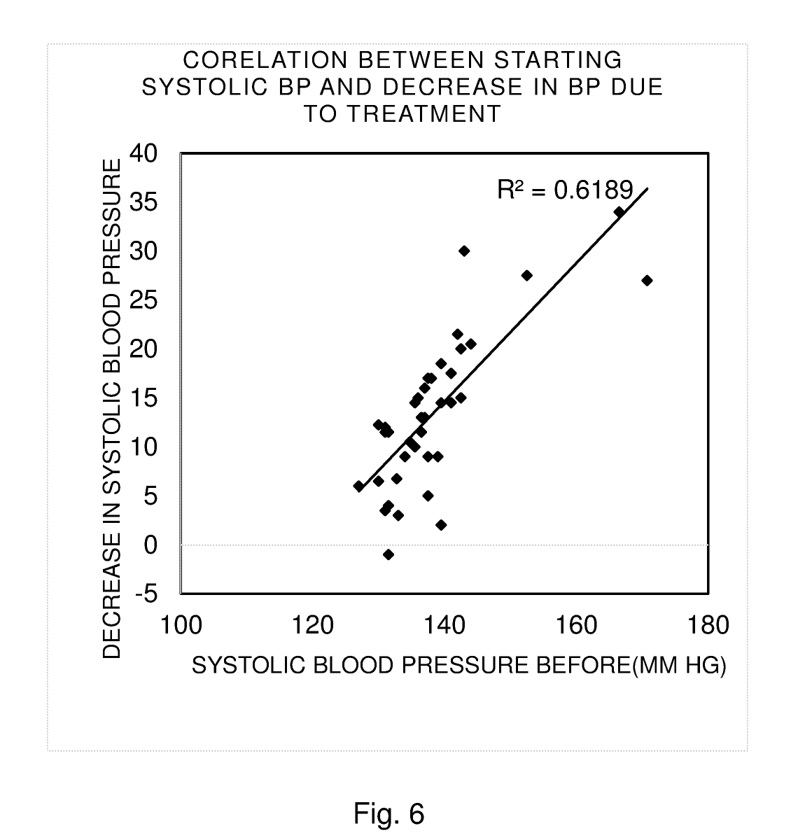

Hence, our vibrational treatment markedly improved the blood pressure of hypertensive individuals (n=26, including 19 already on medication(s)) and basically normalized the blood pressure of pre-hypertensive volunteers, with no negative effect on normotensive individuals. The improvements in test subjects are further illustrated by FIG. 6 that indicates that a line fitted to the data by the least squares method shows greatest benefit to individuals with the highest starting blood pressure.

The observation that there was negligible change in blood pressure in normotensive individuals indicates that normotensive individuals can use the device for other uses. Those uses include, but are not limited to, arrhythmia, PTSD, sleep apnea, depression, and achalasia.

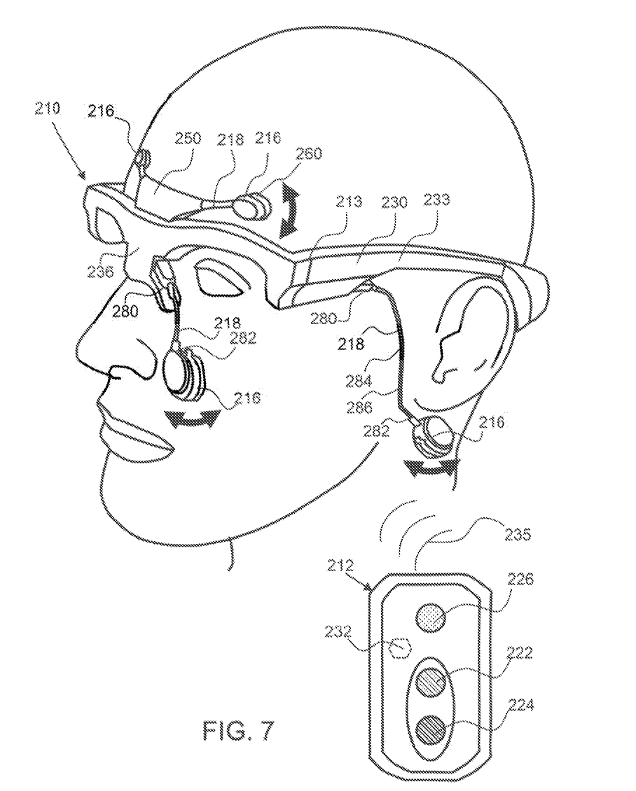

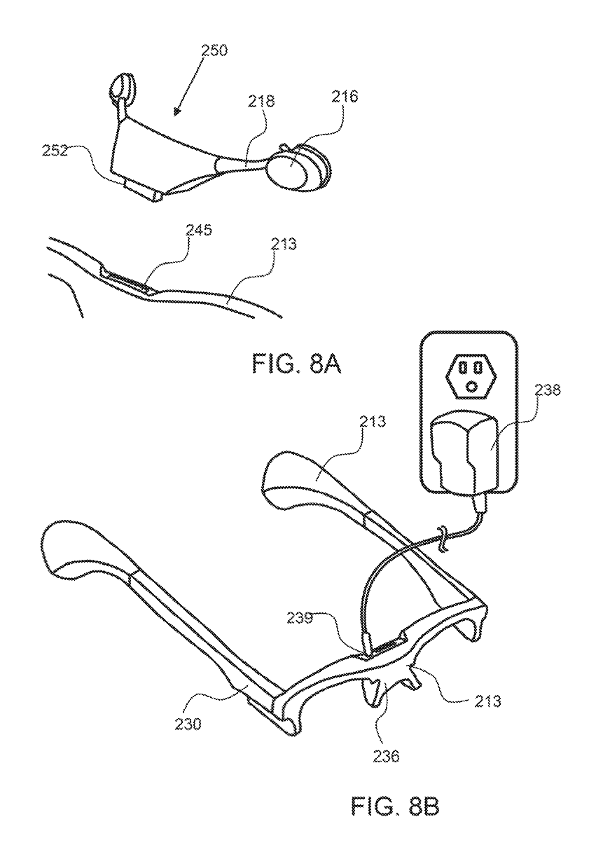

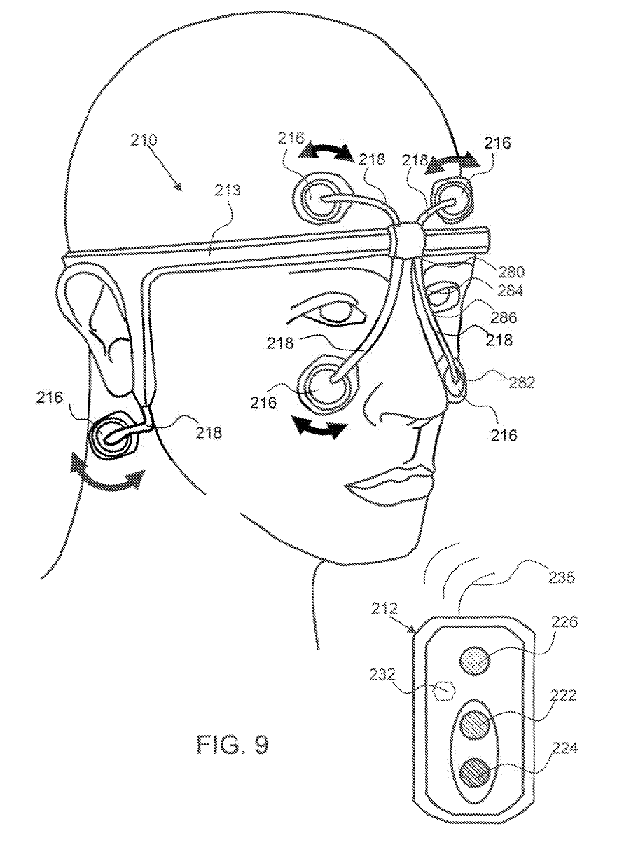

Referring to FIGS. 7 to 9, an exemplary therapeutic vibrational system 210 may have a frame 213 with a plurality (specifically six) of vibrating patches 216-216 connected by flexible tethers 218. In some embodiments the therapeutic vibrational system 210 may have two vibrating patches 216 coupled by tethers to a detachable module 250 having a frame coupling 252 for connecting with the frame 213, and including a frame port 245 as shown in FIGS. 7 and 8A. These vibrating patches 216 may be configured above the frame and coupled to the person's head above or below the eyes. The therapeutic vibrational system 210 may have two vibrating patches 216 coupled to the frame by flexible tethers and configured on the person's face, below the eyes. The therapeutic vibrational system 210 may have two other vibrating patches 216 coupled to the frame by flexible tethers and configured below or behind the person's ears. The flexible tethers 218 have a length from the frame connection end 280 to and the vibrating patch end 282 that enables placement of the vibrating patches 216 in specific locations, as indicated by the bold double arrow curved lines.

The flexible tethers 218 may have a communication line 284 for transferring instructions or commands from the frame 213 to the vibrating patches. The flexible tethers may have a power line 286, for providing electrical power to the vibrating patch to power the vibrating motor. The frame 213 may be an eyeglasses type frame, having extensions from a face portion that extend back and over the ears. The face portion of the frame 213 may have nose interface for resting on the person's nose. The frame may have a frame controller 233 for receiving a wireless signal 235 from a remote controller 212. The remote controller 212 may have a user interface including a frequency control 222, an amplitude control 224 and a program control 226. The amplitude control 224 may control the vibration amplitude of the vibrations created by the vibrating patches. The frequency control 222 may control the frequency of the vibrations created by the vibrating patches. In some embodiments a pulse control turns the vibrating patches on and off, or controls a pulse interval, or a period of time for vibration. A program control 226 may allow a user to select a specific control program that may have preset values for vibration amplitude, frequency, pulse interval and the like. For example, one control program may turn on one or more vibrating patches 216 for a period of time at a frequency and vibration amplitude and then turn on a second vibrating patch at a different frequency and vibration amplitude. In some embodiments, a control program turns on all of the vibrating patches and may change the frequency and vibration amplitude in unison. The remote controller 212 may have a microprocessor 232 and may be a mobile device, such as a cellular phone or tablet computer, for example. As shown in FIGS. 7 and 8B, the battery 230 of the frame 213 may be charged through a charger port 239 by a charger 238. Vibrating patches 216 may include a motor 260, and a signal receiver 236.

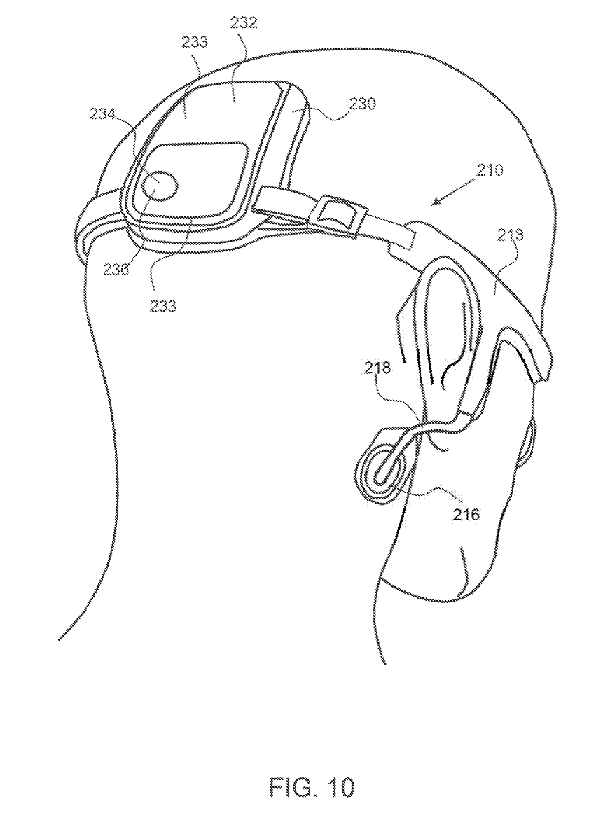

Referring now to FIGS. 7, 9 and 10, an exemplary therapeutic vibrational system 210 comprises a frame 213 with a plurality of vibrating patches 216 connected by flexible tethers 218. The flexible tethers 218 allow placement of the vibrating patches 216 in specific and desired locations, allowing for individual anatomical variations. Each of the tethers 218 may have a length from a frame end 280 to the vibrating patch end 282. The vibrating patches 216 may be moved in any desirable location by moving, bending and rotating the flexible tethers 218, as indicated by the bold double arrow lines. Each of the tethers 218 may have a communication line 284 and a power line 286 for providing electrical power to the patches. The frame in some embodiments may be a head band type, having a band that forms a ring for configuring around the person's head. As shown in FIG. 10, the frame controller 233 may have a battery 230, a microprocessor 232, a signal receiver 236 and signal transmitter 234. Wireless control signals 235 may be received by the frame controller 233 and then transferred to the vibrating patches 216 through the frame 213 and the tethers 218.

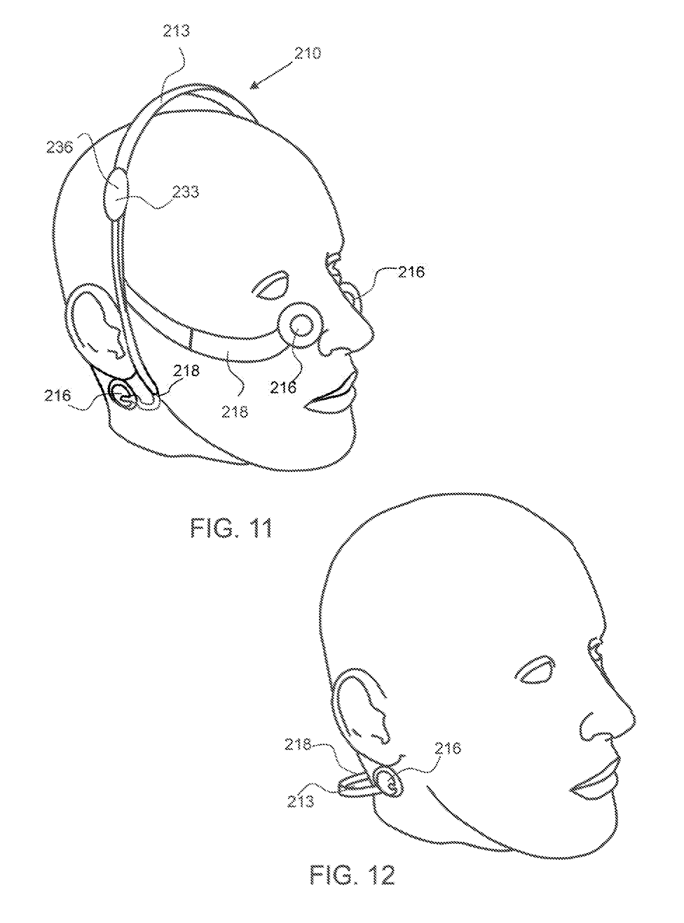

As shown in FIGS. 11 and 12, an exemplary therapeutic vibrational system 210 may have a configuration of frame 213 with a plurality of vibrating patches 216 connected by flexible tethers 218. The frame 213 may be a headset type frame having a flexible arc-shaped open band that can be configured over the person's head, as shown in FIG. 11, or around the person's neck, as shown in FIG. 12.

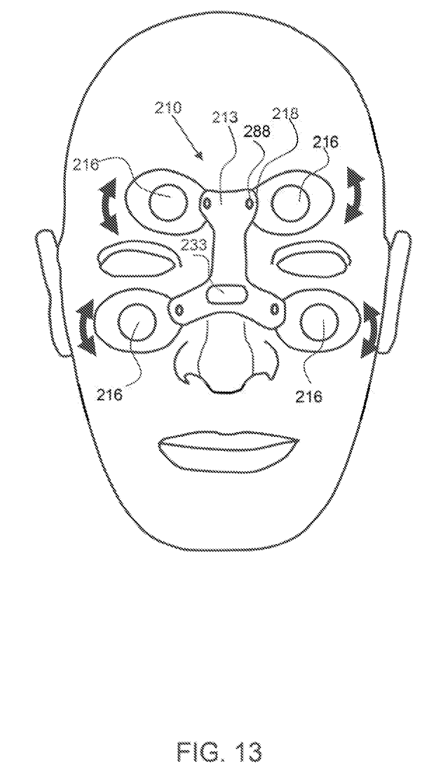

FIG. 13 shows an exemplary therapeutic vibrational system 210 that may have a frame 213 with a plurality of vibrating patches 216 connected by flexible tethers 218. The vibrating patches may be configured to rotate about pivots 288, thereby making their location flexible. The frame 213 may be a nose bridge type frame configured to rest on the bridge of the person's nose.

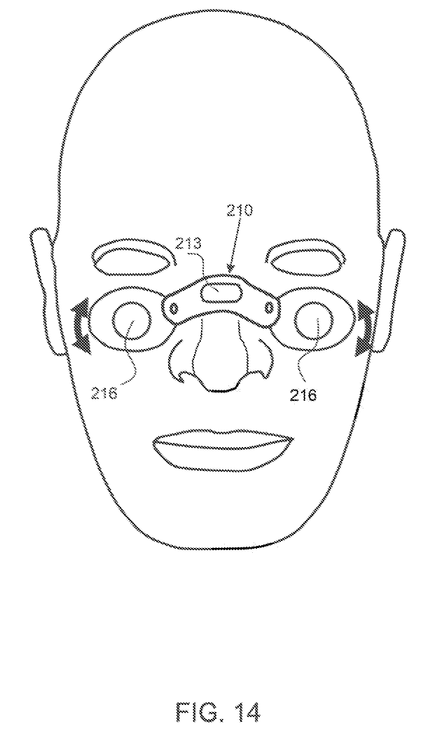

FIG. 14 is a simplified therapeutic vibrational system for ease and comfort. It features two vibrating patches 216 and a frame 213 of the nose bridge type. These may be self-contained patches, including a vibrating motor, a battery, a wireless signal receiver, a wireless signal transmitter and a patch controller. The self-contained patches may be used with a wireless communication such as a phone (not shown).

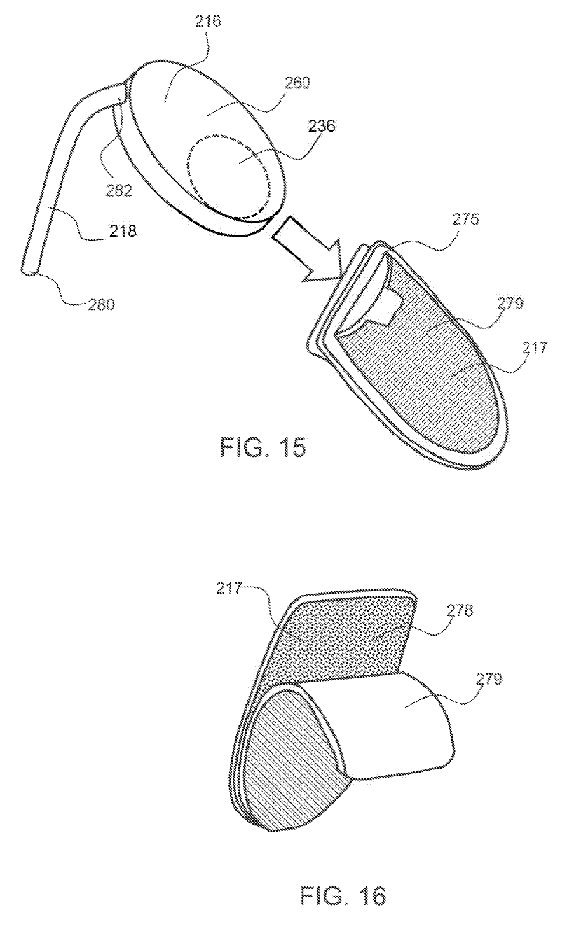

As shown in FIGS. 15 and 16, an exemplary vibrating patch 216 may be inserted into a cover 275 having an opening for receiving the patch and an adhesive patch 217 for attachment to the skin. The adhesive patch 217 may have a removable adhesive cover 279 that when peeled off, exposes the adhesive 278. The covers 275 may be disposable.

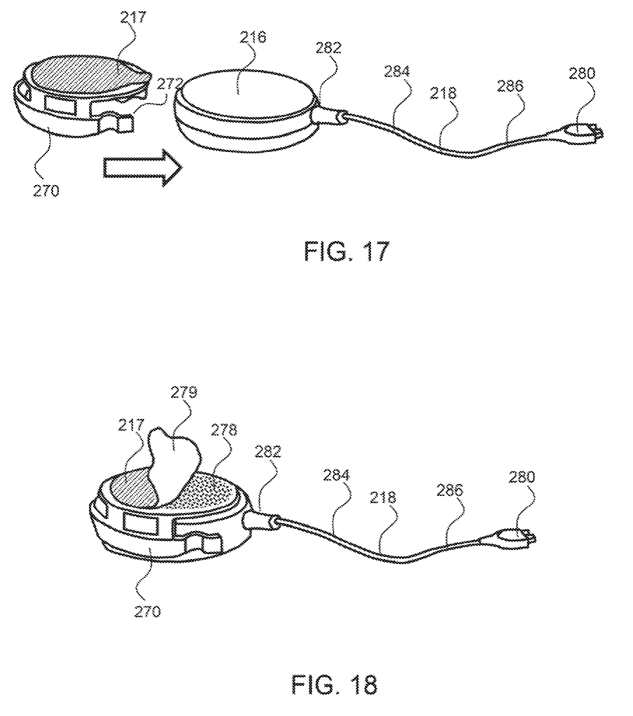

As shown in FIGS. 17 and 18, an exemplary cap 270 may be coupled with a vibrating patch 216. The cap may have an opening 272 with flexible extensions for extending around the vibrating patch and attachment thereto. The cap may have an adhesive patch 217 with a removable adhesive cover 279 that when removed, as shown in FIG. 18, exposes the adhesive 278.

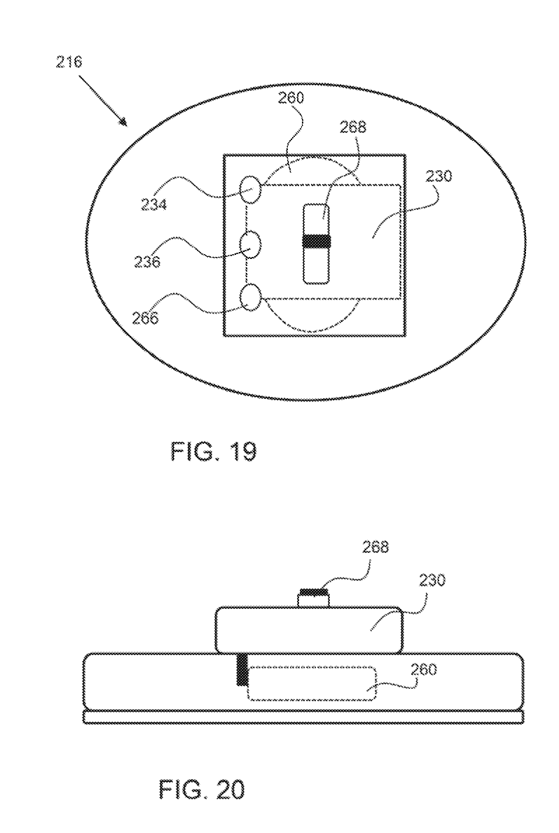

As shown in FIGS. 19 and 20, an exemplary vibrating patch 216 may have a vibrating motor 260, a battery 230, a wireless signal receiver 236, a wireless signal transmitter 234 and a patch controller 266. The vibrating patch 216 may have a tether interface (not shown) for coupling to a flexible tether or to a frame. Vibrating patch 216 in FIG. 19 may be designed to be flatter and may be compatible to be used with clothing. In some embodiments vibrating patch 216 as shown in FIG. 20 may be not as flat but may accommodate more and/or larger electronics. Vibrating patch 216 may include a power switch 268 which may be on top of the outside of the patch.

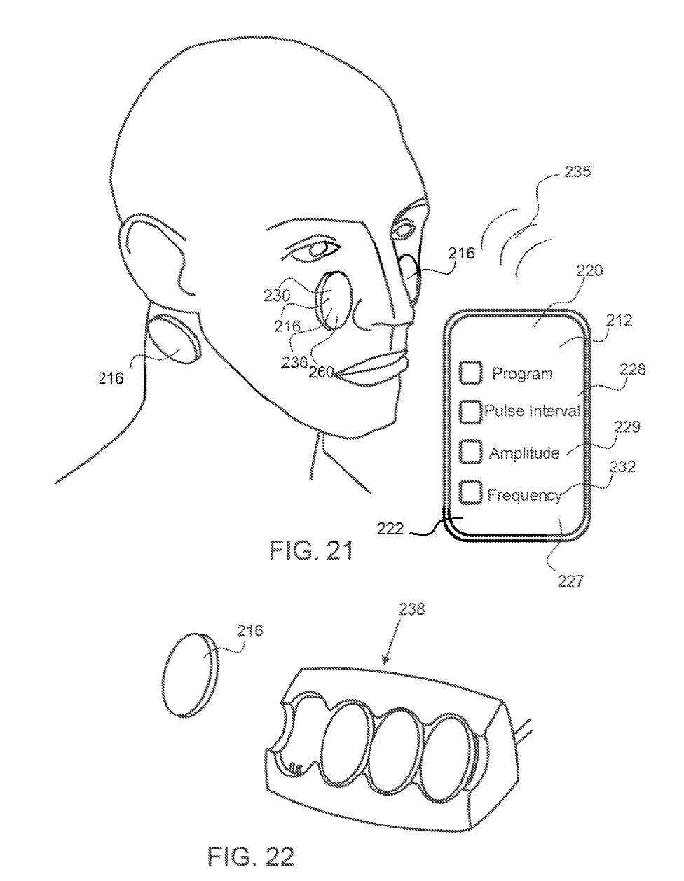

As shown in FIG. 21, a plurality of exemplary vibrating patches 216 may be attached to a person's head (cheek and behind ear). A mobile device controller may have a control program and user interface. The controller 212 may be a mobile device, such as a mobile phone 227 or tablet computer, having a microprocessor 232 and a control program 212. The mobile device may have a wireless signal transmitter which may be used to send wireless signals to the vibrating patches, each having a wireless signal receiver 236. The mobile device may have a user interface 220 with a program control 212, a pulse control 228, an amplitude control 229 and a frequency control 222.

As shown in FIG. 22, an exemplary charger 238 may be configured to receive and charge a plurality of vibrating patches 216.

For remote control on a phone or other internet appliance, a number of features may be provided. For wireless transmission, Bluetooth may be used. After the login/verification process, there may be an opportunity to check for software updates. The main feature tabs may include: settings, treatment, telemed and sync. The device settings may provide device settings for vibration amplitude, duration, programs and playlist. The user setting include but are not limited to user name, user password, age, gender, physician and preferences. The telemed settings can be set to access telemed portal settings. The app settings include turning on or off locations services, integration with contacts and integration with Messaging. Treatment features may include but are not limited to selections for beginning, pausing and ending treatment, as well as blood pressure and/or heart rate readings (provided by a wirelessly connected blood pressure and/or heart rate monitor). The app may enable connection to telemed via an application program interface (API) and ability to select data to be sent. In some embodiments at least two synchronizing procedures may be provided, including synchronizing data with a web portal and synchronizing data with a blood pressure monitor app. Other features may include but are not limited to being able to initiate chat, sending screen shots, accessing a user manual and help, as well as other useful internet links.

It will be apparent to those skilled in the art that various modifications, combinations and variations can be made in the present invention without departing from the spirit or scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

For example, we can provide a self-contained therapeutic vibrational system with at least some of the controls inside the patch. In some embodiments a power switch 268 may be on top of the outside of the patch, in other embodiments a power switch may be internal (pressure sensitive) or a mobile app power control. Moreover, the indicator lights to indicate if the device is on may be positioned on top of the patch or embedded on a circuit board and visible through the patch external material. Such a self-contained patch may still offer wireless communication or communicate by means of a micro USB port on the circuit board.

In some embodiments, the vibrating patch can be held in place with not just the self-adhesive patches but also bands of any stretchy material, such as knits, plastic bands and elastic. The band can lie across the back of the neck and connect two self-contained patches which may be located below or behind the ears. Alternately a band can cross the top of the head and secure two self-contained patches located in front of or below the ears. in some embodiments a band may have two hinged arms which extend to the cheekbones and secure patches beneath the eyes. In another embodiment, a ban crosses the top of the head, with two hinged arms that secure two vibrating patches located in the temple region of the head. In yet another variation, a headband may travel over the forehead and around the back of the head, with a control module residing at the back of the head and its vibrating motors may be contained within disposable caps secured against the skin by memory wire.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

What is claimed is:

1. A therapeutic vibrational system comprising:
a) a plurality of vibrating patches; each comprising:
i) a vibrating motor; and
ii) an adhesive for application to a person's skin;
wherein the plurality of vibrating patches can be placed in two or more locations on said person's skin; and
b) a controller with a user interface and configured to provide control instructions to the plurality of vibrating patches.

2. The therapeutic vibrational system of claim 1, wherein the controller is a remote controller that produces a wireless signal that is received by a wireless signal receiver of each of the plurality of vibrating patches.

3. The therapeutic vibrational system of claim 2, wherein the remote controller is a mobile device.

4. The therapeutic vibrational system of claim 3, wherein the mobile device is a mobile telephone.

5. The therapeutic vibrational system of claim 1, wherein the plurality of vibrating patches are connected to a frame.

6. The therapeutic vibrational system of claim 5, wherein the plurality of vibrating patches are connected to the frame by tethers, wherein the tethers are flexible, allowing adjustable placement of the plurality of vibrating patches with respect to the frame.

7. The therapeutic vibrational system of claim 6, wherein the tethers comprise a communication line for transmission of control instructions from said frame controller to the plurality of vibrating patches.

8. The therapeutic vibrational system of claim 7, wherein the frame controller comprises a user interface.

9. The therapeutic vibrational system of claim 7, wherein the frame controller comprises a wireless signal receiver for receiving a wireless signal having control instructions from a remote controller.

10. The therapeutic vibrational system of claim 9, wherein the remote controller has a user interface having amplitude control and a frequency control.

11. The therapeutic vibrational system of claim 9, wherein the remote controller is a mobile device.

12. The therapeutic vibrational system of claim 11, wherein the mobile device is a mobile telephone.

13. The therapeutic vibrational system of claim 5, wherein the frame comprises a battery and wherein the frame supplies electrical power to the plurality of vibrating patches from said battery.

14. The therapeutic vibrational system of claim 13, wherein the plurality of vibrating patches are connected to the frame by tethers; wherein the tethers are flexible, allowing adjustable placement of the plurality of vibrating patches with respect to the frame.

15. The therapeutic vibrational system of claim 14, wherein the tethers comprise a power line for providing electrical power from frame battery to each of the plurality of vibrating patches.

16. The therapeutic vibrational system of claim 15, wherein the tethers comprise a communication line for transmission of control instructions from said frame controller to the plurality of vibrating patches.

17. The therapeutic vibrational system of claim 14, wherein the frame controller comprises a wireless signal receiver for receiving a wireless signal having control instructions from a remote controller.

18. The therapeutic vibrational system of claim 16, wherein the remote controller is a mobile device.

19. The therapeutic vibrational system of claim 1, comprising a cover having an adhesive patch, wherein the cover comprises an opening for receiving one of said plurality of vibrating patches.

20. The therapeutic vibrational system of claim 1, comprising a detachably attachable cap having an adhesive patch, wherein the cap comprises an opening for detachably attachment to one of said plurality of vibrating patches.

21. A therapeutic vibrational system comprising:
a) a frame comprising:
i) a frame controller;
ii) a wireless signal receiver for receiving control instructions; and
iii) a battery;
b) a plurality of vibrating patches, each comprising:
i) a vibrating motor; and
ii) an adhesive for application to a person's skin;
wherein the plurality of vibrating patches can be place in two or more locations on said person's skin; and
c) a plurality of tethers coupling the plurality of vibrating patches to the frame, each of said tethers comprising:
i) a communication line for transmission of said control instructions from said frame controller to the plurality of vibrating patches; and
ii) a power line for providing electrical power from the frame battery to each of the plurality of vibrating patches;
wherein the wireless signal receiver of the frame receives control instructions from a remote controller having a user interface.

22. The therapeutic vibrational system of claim 21, wherein the remote controller is a mobile device.

23. The therapeutic vibrational system of claim 22, wherein the mobile device is a mobile telephone.

24. The therapeutic vibrational system of claim 22, wherein the remote controller has a user interface having amplitude control and a frequency control.

25. The therapeutic vibrational system of claim 21, having at least four vibrating patches.

26. The therapeutic vibrational system of claim 21, wherein the frequency of the vibrating patches is 20-500 Hz.
